(19) United States
(12) Patent Application Publication
(10) Pub. No.: US 2004/0048890 A1
(43) Pub. Date: Mar. 11, 2004

(54) Compositions comprising a leukotriene inhibitor and a decongestant

(76) Inventor: Steven A. Nichtberger, Villanova, PA (US)

Correspondence Address: Mollie M. Yang, Merck & Co., Inc., Patent Dep., RY60-30, P.O. Box 2000, Rahway, NJ, 07056-0907, US

(21) Appl. No.: 10/643,378

(22) Filed: Aug. 19, 2003

Related U.S. Application Data
(60) Provisional application No. 60/408,515, filed on Sep. 5, 2002.

(51) Int. Cl. A61K 31/47

(52) U.S. Cl. 514/311

(57) ABSTRACT

The present invention provides oral pharmaceutical compositions comprising a leukotriene inhibitor and pseudoephedrine for the treatment of allergies, asthma and associated signs and symptoms.

### BACKGROUND OF THE INVENTION

Leukotriene inhibitors such as 5-lipooxygenase inhibitors and leukotriene receptor antagonists have been established as effective treatment of asthma, and some of these agents are also being investigated, or have been approved, for the treatment of allergic rhinitis. Decongestants are effective in relieving nasal congestion in patients with that symptom. The combination of a leukotriene inhibitor and a decongestant will provide for more complete and/or more effective and/or more rapid relief of the congestion and other signs and symptoms of asthma or allergic rhinitis.

### SUMMARY OF THE INVENTION

The present invention relates to pharmaceutical compositions comprising a leukotriene inhibitor in combination with a decongestant for the treatment of allergies and asthma.

### DETAILED DESCRIPTION OF THE INVENTION

The present invention provides for novel oral pharmaceutical compositions comprising as pharmaceutically active compounds a combination of an antiallergy-effective amount of a leukotriene inhibitor or a pharmaceutically acceptable salt thereof, and of a decongestant-effective amount of pseudoephedrine or a pharmaceutically acceptable salt thereof, and further comprising pharmaceutically acceptable carriers or excipients with the proviso that the compositions do not contain an antihistamine.

In another aspect the present invention provides a method for treating allergies which comprises administering to a patient in need of such treatment a pharmaceutical composition of the present invention. The pharmaceutical compositions of the present invention can also include an additional active ingredient selected from antitussives, expectorants, mucolytics, and analgesic-antipyrectics. Antitussives are for example dextromethorphan, codeine, terpin hydrate and pharmaceutically acceptable salts thereof. Expectorants include, but are not limited to, guaifenesin, potassium guaicolsulfonate, potassium iodide, potassium citrate, iodinated glycerol, acetylcysteine, carboxymethylcysteine, ambroxol, sobrerol, and pharmaceutically acceptable salts thereof. Mucolytics are, for example, bromhexine and ambroxol. Analgesic-antipyretic compounds include, but are not limited to, acetylsalicylic acid, acetaminophen, ketoprofen, naproxen and ibuprofen.

The term “leukotriene inhibitor” as used herein includes any agent or compound that inhibits, restrains, retards or otherwise interacts with the action or activity of leukotrienes, such as, but not limited to, 5-lipoxygenase (“5-LO”) inhibitors, 5-lipoxygenase activating protein (“FLAP”) antagonists, and leukotriene receptor antagonists (“LTRAs”).

The term “5-lipoxygenase inhibitor” or “5-LO inhibitor” as used herein includes any agent or compound that inhibits, restrains, retards or otherwise interacts with the enzymatic action of 5-lipoxygenase, such as, but not limited to, zileuton (ZYFLO®), docebenone, piripost, CI-D2318, ZD4407 and CJ13610 (4-(3-[4-(2-methyl-imidazol-1-yl)-phenylsulfanyl]phenyl)tetrahydropyran-4-carboxylic acid amide).

The term “5-lipoxygenase activating protein antagonist” or “FLAP antagonist” as used herein includes any agent or compound that inhibits, restrains, retards or otherwise interacts with the action or activity of 5-lipoxygenase activating protein, such as, but not limited to, MK-591 and MK-886.

The term “leukotriene receptor antagonist” or “LTRA” as used herein includes any agent or compound that inhibits, restrains, retards or otherwise antagonizes the activity of receptors that are responsive to leukotrienes, including those responsive to leukotriene D₄. Exemplary LTRAs include sodium 1-(((R)-(3-(2-(6,7-difluoro-2-quinolinyl)ethenyl)phenyl)-3-(2-(2-hydroxy-2-propyl)phenyl)thio)methyl)cyclopropaneacetate, 1-(((1 (R)-(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid and salts thereof, pranlukast, zafirlukast (ACCOLATE®), and montelukast sodium (MK-476, SINGULAIR®).

The term “pharmaceutically acceptable salt” refers to a salt prepared from pharmaceutically acceptable non-toxic acids or bases including inorganic acids or bases or organic acids or bases. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, pamoic, methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), lysine and procaine.

The term “antiallergy-effective amount” as used herein means that amount of a leukotriene inhibitor, or a pharmaceutically acceptable salt thereof, which, alone or in combination with other drugs, provides a therapeutic benefit in the treatment, management, control or prevention of signs and symptoms associated with allergies.

The term “decongestant-effective amount” as used herein means that amount of decongestant which alone, or in combination with other drugs, provides a therapeutic benefit in the treatment, management, or prevention of congestion of the respiratory tract and/or sinus.

The term “allergies” means diseases or disorders associated with Type I hypersensitivity reactions. Examples of allergic conditions contemplated include allergic rhinitis (seasonal or perennial, and including allergic rhinitis coexisting with asthma), allergic conjunctivitis, allergic asthma and urticaria.

The term “treatment” or “treating” includes alleviating, ameliorating, relieving or otherwise reducing, as well as preventing onset of symptoms commonly associated with allergic conditions.

The leukotriene inhibitors used in the present pharmaceutical compositions are known in the art. For example, zileuton is described in U.S. Pat. No. 4,873,259; zafirlukast is described in U.S. Pat. No. 4,859,692, and montelukast is described in U.S. Pat. No. 5,565,473. In one embodiment of the present pharmaceutical compositions, the leukotriene inhibitor component is a leukotriene receptor antagonist. In one subset thereof the leukotriene receptor antagonist is montelukast or a pharmaceutically acceptable salt thereof, and preferably it is montelukast sodium. Pseudoephedrine is preferably used as the hydrochloride or the sulfate.

The pharmaceutical compositions according to the present invention are useful for treating a patient suffering from the signs and symptoms associated with upper respiratory disease or asthma and nasal congestion. The combination of a leukotriene inhibitor and a decongestant will provide for more complete and/or more effective and/or more rapid relief of the congestion and other signs and symptoms of asthma or allergic rhinitis. Furthermore the compositions according to the invention are useful in the treatment of, for instance, common cold and in the symptomatic relief associated with cough, cold and flu symptoms.

The magnitude of a prophylactic or therapeutic dose of leukotriene inhibitor in the acute or chronic prevention, treatment, or management of a disorder or condition will vary with the severity of the condition to be treated, the particular leukotriene inhibitor chosen, and patient characteristics such as age, body weight, and response of the individual patient. Suitable total daily dose ranges can be readily determined by those skilled in the art. For example, for 5-lipoxygenase inhibitors, a preferred oral daily dose range of leukotriene inhibitor is typically from about 20 mg to 2,500 mg, preferably about 20 mg to 800 mg. For leukotriene receptor antagonists, a preferred oral daily dose is typically from about 2 mg to 100 mg, preferably about 4 mg to 20 mg. A preferred oral daily dose range of pseudoephedrine is from about 50 mg to about 300 mg, more preferably, about 150 mg to about 250 mg. The pharmaceutical composition of the present invention may be administered one to four times daily; preferably once or twice daily.

It is further recommended that children, patients aged over 65 years, and those with impaired renal or hepatic function initially receive low doses, and that they then be titrated based on individual response(s) or blood level(s). It may be necessary to use dosages outside these ranges in some cases as will be apparent to those skilled in the art. Further, it is noted that the clinician or treating physician will know how and when to adjust, interrupt, or terminate therapy in conjunction with individual patient response.

Pharmaceutical compositions of the present invention are produced by formulating the active compounds in unit dosage form with a pharmaceutical carrier. Some examples of dosage unit forms are tablets including film-coated tablets and chewable tablets, capsules including hard- and soft gelatin capsules, cachets, pills, powders, and aqueous and nonaqueous oral solutions and suspensions packaged in containers containing either one or some larger number of dosage units and capable of being subdivided into individual doses. Some examples of suitable pharmaceutical carriers, including pharmaceutical diluents, are gelatin capsules; sugars such as lactose and sucrose; starches such as corn starch and potato starch, cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations. The compositions of the invention can also contain other components such as coloring agents, flavoring agents, and/or preservatives. These materials, if present, are usually used in relatively small amounts. The compositions can, if desired, also contain other therapeutic agents. Because of their ease of administration, tablets and capsules represent the most advantageous oral dosage unit form, in which case solid pharmaceutical carriers are employed. If desired, tablets may be coated by standard aqueous or nonaqueous techniques.

Pharmaceutical compositions of the present invention may be prepared by any of the methods of pharmacy, but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. Desirably, each tablet contains from about 1 mg to about 500 mg of each active ingredient, and each cachet or capsule contains from about 1 mg to about 500 mg of each active ingredient. Preferably, the tablet, cachet or capsule contains any one of four dosages, 4 mg, 5 mg, 10 mg or 20 mg of montelukast sodium as the leukotriene inhibitor and any one of three dosages 60 mg, 120 mg or 240 mg of pseudoephedrine hydrochloride or sulfate.

The amounts of the active ingredient(s) to achieve therapeutic effects will vary, depending on the activities of the specific compounds used, the specific disease to be treated, the severity of the disease, and the conditions of the patients to be treated. The dose for each active compound may be one usually used when the drug is administered alone, or it may be lower than such usual dose as the combination of the active ingredients may be synergistic for the treatment of the target diseases. Generally the dose may be between about 1 and about 1000 milligrams of each compound administered in a dose. The compounds may be combined in a single dosage formulation, or may be administered in separate dosage forms, and these may be solid (such as tablets, capsules, sachets and the like) or liquid (such as solutions or suspensions).

The weight ratio of the leukotriene inhibitor to the pseudoephedrine may be varied and will depend upon the effective dose of each ingredient. Generally, an effective dose of each will be used. Thus, for example, when a leukotriene inhibitor is combined with pseudoephedrine the weight ratio of the former to the latter will generally range from about 1:100 to about 100:1, preferably about 1:10 to about 1:50.

In one embodiment the present invention relates to an oral pharmaceutical composition providing for a sustained release of the decongestant effective amount of pseudoephedrine and an immediate release of an antiallergy-effective amount of the leukotriene inhibitor. Such composition may be, for example, a bilayer tablet wherein a first layer A, providing for the sustained release of pseudoephedrine comprises a decongestant effective amount of pseudoephedrine or a pharmaceutically acceptable salt thereof and wherein a second layer B, providing for the immediate release of the leukotriene inhibitor component comprises an antiallergy effective amount of a leukotriene inhibitor or a pharmaceutically acceptable salt thereof. The bilayer tablet according to the invention may additionally contain a tablet coating C consisting of pharmaceutically acceptable excipients which mask the taste of one of the active compounds.

In a bilayer tablet, layer A of said tablet comprises a decongestant effective amount of pseudoephedrine or a pharmaceutically acceptable salt thereof in a matrix of a swellable hydrophilic polymer which provides a sustained release profile in a period of 3 to 24, preferably 6 to 12, most preferably about 12 hours to about 24 hours.

The concentration range of pseudoephedrine salt in the compositions according to the invention is between 5 and 240 mg/tablet, preferably 10 to 200 mg/tablet, more preferably 60 to 240 mg/tablet. The concentration range of the leukotriene inhibitor in the compositions according to the invention is between 2 and 500 mg/tablet, preferably 4 to 20 mg/tablet.

Each layer of the tablet is in contact with each other in a portion of their surface, but provides independent release profiles for both active substances mentioned before. The sustained release layer A consists of pseudoephedrine or a pharmaceutically acceptable salt thereof and a swellable hydrophilic polymer. Typical swellable hydrophilic polymers include cellulosic ethers such as methylcellulose, ethylcellulose, hydroxypropylcellulose, hydroxypropylmethylcellulose, hydroxymethylcellulose, hydroxyethylcellulose, carboxymethylcellulose and carboxyethylcellulose or mixtures thereof. The use of hydroxypropylmethylcellulose (HPMC) is preferred. Suitable HPMC polymers are, for example, HPMC USP2910 and USP2208 such as Methocel E5, E4M, E15M, K15M, and K100M supplied by the Dow Chemical Company. In the aforementioned abbreviations the designation “E” refers to USP2910 whereas “K” refers to USP2208. The number designation refers to the viscosity in a 2% aqueous solution (e.g. 5 designates a viscosity of 5 cps; 15M designates a viscosity of 15000 cps)

The excipients that could be optionally used in the sustained release layer A are insoluble polymers, soluble or insoluble fillers, antiadherents, coloring agents, lubricants and additional binders. Typical fillers are for example lactose, microcrystalline cellulose, dibasic calcium phosphate and cornstarch. Examples of antiadherents, which are used to prevent tablets from sticking to the tablet press, are colloidal silicon dioxide and talc. Magnesium stearate, talc and stearic acid are typical lubricants. Typical binders are povidone, and cornstarch.

The immediate release matrix layer B comprises the leukotriene inhibitor within different combinations of excipients. The excipients that could be optionally used in the immediate release layer B are insoluble polymers, soluble or insoluble fillers, antiadherents, lubricants, coloring agents, disintegrants and additional binders. Typical fillers are for example lactose, microcrystalline cellulose, dibasic calcium phosphate and cornstarch. Examples of antiadherents, which are used to prevent tablets from sticking to the tablet press, are colloidal silicon dioxide and talc. Typical disintegrants are crospovidone, sodium starch glycolate and crosscarmellose sodium. Typical coloring agents are selected from FD& C red 40 HT Aluminum lake, 2-hydroxy-1,1′-azonaphthalene-3,6,4′-trisulfonic acid trisodium salt, erythrosine, iron oxides, 1-(4-sulpho-1-naphthylazo)-2-naphthol-6,8-disulphonic acid trisodium salt, 2′,4′,5′,7′-tetrabromo-4,5,6,7-tetrachloro-fluorescein disodium salt, 2,4,5,7-Tetraiodo-3,6-dihydroxyxanthene-9-spiro-1′-(4′,5′,6′,7′-tetrachloro-3′H-isobenzofuran-3′one dipotassium salt, trisodium 3-carboxy-5-hydroxy-1-p-sulphophenyl-4-p-sulfophenylazopyrazole, 6-hydroxy-5-((4-sulphonphenyl) azo-2-naphthalenesulphonic acid disodium salt and optionally aluminium lakes thereof. Magnesium stearate, talc and stearic acid are typical lubricants. Typical binders are povidone, and cornstarch.

Water and ethanol are examples of volatile components which can be used in the manufacture process of both layers to granulate powders. These volatile components are removed during processing and therefore do not appear in the finished product.

The tablet coating is optional since the presence of it does not modify significantly the release rates of the active substances present in the core layers. The presence of the coating is preferred because it may mask the unpleasant taste of one of the active substances and enhance the properties of dosage form. Different coatings with different polymers, and plasticizers and other excipients may be used without significantly affecting the release profile of the active substances present in the core tablet. A typical coating comprises a polymer such as hydroxypropylmethylcellulose and a plasticizer such as polyethylene glycol. Optional excipients could be added to the coating like antifoaming agents and opacifying agents. Example of an antifoaming agent is silicone. Examples of opacifying agents are titanium dioxide, talc and aluminum lake dyes.

The invention will be further described by the following examples. These examples disclose certain preferred embodiments of the invention. The methods of manufacturing the compositions according to the invention such as granulation, tablet compression, tablet coating etc. are well known to the person skilled in the art. Those skilled in the art will appreciate that various changes, modifications and substitutions can be made therein without departing from the spirit of the invention. Accordingly, it is intended that the invention be not limited to the following explicitly disclosed examples.

### EXAMPLE 1

CORE

mg/tablet

A. First Layer

| | |
|---|---|
| Layer pseudoephedrine | |
| Pseudoephedrine_sulfate | 120.00 |
| Methocel K 15 M PRCR* | 198.00 |
| Lactose Monohydrate | 105.10 |
| Microcrystalline cellulose | 106.00 |
| Colloidal silicon dioxide | 1.65 |
| Magnesium Stearate | 2.75 |
| Povidone | 16.50 |
| Total first layer | 550.00 |

B. Second layer

| | |
|---|---|
| Layer leukotriene inhibitor | |
| montelukast sodium | 10.00 |
| FD&C red 40 HT Aluminum lake (allura red AC) | 0.38 |
| Microcrystalline cellulose | 70.00 |
| Lactose Monohydrate | 154.62 |
| Povidone | 12.50 |
| Magnesium Stearate | 2.50 |
| Total second layer | 250.00 |
| Total core | 800.00 |

C. Coating

| | |
|---|---|
| Film Coating | |
| Methocel E5 | 15.00 |
| Polyethylene Glycol 6000 | 1.97 |
| Silicone antifoam S184 | 0.03 |
| Total film coating | 17.00 |
| Total Film coated tablet | 817.00 |

*PR means Premium grade and CR means Controlled Released grade.

Method of Manufacture

A. First Layer:

A1. Dissolve povidone in a hydroalcoholic mixture;

A2. Blend pseudoephedrine sulfate, a portion of the microcrystalline cellulose, lactose and Methocel K15M for 5-30 minutes in a suitable mixer.

A3. Use alcoholic or hydroalcoholic solution prepared previously in step A1. to granulate the powder mix.

A4. Dry and mill the pseudoephedrine sulfate granulation from step A3, using suitable size screen.

A5. Blend the screened pseudoephedrine sulfate granulation with a portion of the microcrystalline cellulose and colloidal silicon dioxide for 3-15 minutes.

A6. Add magnesium stearate and blend for 3-15 minutes.

B. Second Layer:

B1. Pass through a suitable screen montelukast sodium, Allura red AC (FD & C red 40 HT) aluminum lake and microcrystalline cellulose. Blend for 5-30 minutes in a suitable mixer.

B2. Add lactose and povidone. Blend for 60 minutes 15-120 minutes in a suitable mixer.

B3. Add magnesium stearate. Blend for 3-20 minutes in a suitable mixer.

C. Compression:

Compress A and B into a suitable bilayer tableting machine in suitable size tablets.

D. Coating

D1. Dissolve Methocel E5 and Polyethylene Glycol in suitable amount of water.

D2. Dissolve silicone antifoam in suitable amount of isopropyl alcohol.

D3. Add 2. to 1. and mix.

D4. Coat tablets with the Methocel E5/Polyethylene glycol solution from step D3 in a suitable coater.

### EXAMPLE 2

CORE

mg/tablet

A. First Layer

| | |
|---|---|
| Layer pseudoephedrine | |
| Pseudoephedrine_sulfate | 120.00 |
| Methocel K 15 M PRCR* | 198.00 |
| Lactose Monohydrate | 126.50 |
| Microcrystalline cellulose | 100.00 |
| Colloidal silicon dioxide | 2.75 |
| Magnesium Stearate | 2.75 |
| Total first layer | 550.00 |

B. Second layer

| | |
|---|---|
| Layer leukotriene inhibitor | |
| montelukast sodium | 10.00 |
| Punceau 4R red aluminum lake | 0.38 |
| Microcrystalline cellulose | 70.00 |
| Lactose Monohydrate | 168.40 |
| Magnesium Stearate | 1.25 |
| Total second layer | 250.00 |
| Total core | 800.00 |

C. Coating

| | |
|---|---|
| Film Coating | |
| Methocel E5 | 4.42 |
| Polyethylene Glycol 6000 | 2.72 |
| Talc | 8.76 |
| Titanium dioxide | 1.10 |
| Total film coating | 17.00 |
| Total Film coated tablet | 817.00 |

*PR means Premium grade and CR means Controlled Released grade.

Method of Manufacture

First Layer:

A1. Blend pseudoephedrine sulfate, microcrystalline cellulose, lactose, colloidal silicon dioxide and HPMC K15M for 5-30 minutes in a suitable mixer.

A2. Add magnesium stearate and blend for 3-15 minutes.

B. Second Layer:

B1. Pass through a suitable screen monteluksast sodium, and microcrystalline cellulose. Blend for 5-30 minutes in a suitable mixer.

B2. Add lactose. Blend for 60 minutes 15-120 minutes in a suitable mixer.

B3. Add magnesium stearate. Blend for 3-20 minutes in a suitable mixer.

C. Compression:

Compress A and B into a suitable bilayer tableting machine in suitable size tablets.

D. Coating

D1. Dissolve Methocel E5 and Polyethylene Glycol in suitable amount of water.

D2. Add Titanium Dioxide and Talc in suitable amount of water and mix

D3. Add 2. to 1. And mix.

D4. Coat tablets with the Methocel E5/Polyethylene glycol solution from step D3. in a suitable coater.

### EXAMPLE 3

CORE
A. First Layer

| Layer pseudoephedrine | mg/tablet |
|---|---|
| Pseudoephedrine_sulfate | 120.00 |
| Methocel K4M | 247.50 |
| Lactose Monohydrate | 166.0 |
| Talc | 11.00 |
| Magnesium Stearate | 5.50 |
| Total first layer | 550.00 |

Second layer and coating are identical to example 2; the manufacture method was conducted analogously to the method outlined in example 2.

### EXAMPLE 4

CORE
A. First Layer

| Layer pseudoephedrine | mg/tablet |
|---|---|
| Pseudoephedrine_sulfate | 120.00 |
| Methocel K 15 M PRCR | 198.00 |
| Lactose Monohydrate | 99.50 |
| Microcrystalline cellulose | 99.50 |
| Colloidal silicon dioxide | 2.75 |
| Magnesium Stearate | 2.75 |
| Povidone | 27.50 |
| Total first layer | 550.00 |

Second layer and coating are identical to example 1; the manufacture method was conducted analogously to the method outlined in example 1.

### EXAMPLE 5

CORE
A. First Layer

| Layer pseudoephedrine | mg/tablet |
|---|---|
| Pseudoephedrine_sulfate | 120.00 |
| Methocel K 15 M CR | 137.50 |
| Methocel K100M CR | 137.50 |
| Lactose | 138.50 |
| Talc | 11.00 |
| Magnesium Stearate | 5.50 |
| Ethanol | s.q. |
| Total first layer | 500.00 |

Second layer and coating are identical to example 1; the manufacture method was conducted analogously to the method outlined in example 1.

What is claimed is:

1. An oral pharmaceutical composition comprising as pharmaceutically active compounds a combination of an antiallergy-effective amount of a leukotriene inhibitor or a pharmaceutically acceptable salt thereof, and of a decongestant-effective amount of pseudoephedrine or a pharmaceutically acceptable salt thereof, and further comprising pharmaceutically acceptable carriers or excipients with the proviso that the compositions do not contain an antihistamine.

2. A composition of claim 1 wherein said leukotriene inhibitor is a leukotriene receptor antagonist.

3. A composition of claim 2 wherein said leukotriene receptor antagonist is montelukast or a pharmaceutically acceptable salt thereof.

4. A composition of claim 2 wherein said leukotriene receptor antagonist is montelukast sodium.

5. A composition of claim 1 in solid dosage form.

6. A composition of claim 5 wherein said dosage form is a tablet.

7. A composition of claim 6 wherein said tablet contains montelukast sodium in an amount selected from 4 mg to 20 mg, and pseudoephedrine or a pharmaceutically acceptable salt thereof in an amount selected from 60 mg to 240 mg.

8. A composition of claim 6 wherein said tablet contains montelukast sodium in an amount selected from 4 mg, 5 mg, 10 mg and 20 mg, and pseudoephedrine or a pharmaceutically acceptable salt thereof in an amount selected from 60 mg, 120 mg and 240 mg.

9. A method for the treatment of allergic rhinitis or asthma which comprises administering to a patient in need of such treatment a pharmaceutical composition of claim 1.
